(19) United States
(12) Patent Application Publication
(10) Pub. No.: US 2012/0021969 A1
(43) Pub. Date: Jan. 26, 2012

(54) PROSTHETIC IMPLANT SHELL

(75) Inventors: Cornelia C. Haag-Molkenteller, Irvine, CA (US); Janet K. Cheetham, Laguna Niguel, CA (US)

(73) Assignee: ALLERGAN, INC., Irvine, CA (US)

(21) Appl. No.: 13/254,393

(22) PCT Filed: Mar. 3, 2010
(86) PCT No.: PCT/US10/25992

§ 371 Date: Oct. 14, 2011

Related U.S. Application Data
(60) Provisional application No. 61/158,277, filed on Mar. 6, 2009.

(51) Int. Cl. A61K 38/16; A61P 15/08; A61P 15/00

(52) U.S. Cl. 514/1.1

(57) ABSTRACT

Methods for improving ejaculate of a patient in need thereof are presented, more particularly, methods for increasing ejaculate volume/prostatic fluid to increase viability of sperm contained therein by administration of a neurotoxin to the prostate of the patient, are provided.

### CROSS-REFERENCE

This application claims the benefit of U.S. Provisional Patent Application Ser. No. 61/158,277, filed on Mar. 6, 2009, the entire disclosure of which is incorporated herein by this specific reference.

### BACKGROUND

Methods for improving ejaculate of a patient in need thereof are presented. More particularly, methods for increasing ejaculate volume/prostatic fluid to increase viability of sperm contained therein, by administration of a neurotoxin to the prostate of the patient are provided.

As is well known, the prostate in human males is located beneath the bladder and ventral to the rectum, encircling a section of the urethra. During ejaculation, prostatic fluid (semen) is secreted by the prostate through small pores of the urethra's walls, which then carry mature sperm out of the body.

The prostate is made up of three lobes, encased by an outer layer, which is flanked on either side by the seminal vesicles. Next to the seminal vesicles run the two vas deferens, tubes that carry sperm from the testicles. These vasa deferrentia pass directly behind the prostate and open into to urethra to propel sperm out into the urethra, in the prostatic section of the urethra. The prostate is divided into the anterior lobe (anterior portion of the gland lying in front of the urethra) median lobe (the cone-shaped portion of the prostate located between the two ejaculatory ducts and the urethra) and the lateral lobes (right and left, separated by the prostatic urethra) that form the main mass of the gland and are continuous posteriorly. The posterior lobe is the postero-medial part of the lateral lobes which is palpated through the rectum during a digital rectal exam.

The prostate plays an important role in the excretion of a prostatic fluid contributing to the overall seminal fluid. The prostatic fluid contributes to about 10-30% of the seminal fluid of an ejaculate. It is mainly composed of proteolytic enzymes, prostate acid phosphatase, and zinc in very high concentrations. Due to its alkaline pH, it is proposed that the prostatic fluid component of the overall seminal fluid protects the sperm against the acidic milieu of the vagina. In addition, the high zinc content may provide additional nutrients contributing to sperm motility. Thus, it can seen that prostatic fluid and its contribution to and as part of the ejaculate play an important part in providing an appropriate milieu conducive to sperm health, including sperm motility and overall condition of the ejaculate, that is, facilitating the overall success of fertilizing an egg. Infertility is typically considered as the inability to conceive after at least one year of unprotected intercourse. Infertility in men can be cause by hormone disorders, illness, reproductive anatomy trauma, obstruction and sexual dysfunction, among other reasons. According to the National Institutes of Health, male infertility is involved in approximately 40% of the 2.6 million infertile married couples in the United States. Common causes for male infertility include impaired sperm production and/or impaired sperm delivery. Investigating a male's fertility typically includes a thorough examination and review of the male's medical and surgical history, which includes a semen analysis and examination of ejaculate, as seminal fluid can affect sperm function and movement, as discussed above. Sperm factors typically considered include concentration (sperm/milliliter), morphology, motility (% sperm movement), testing of thickness and/or color of ejaculate, total motile sperm count and volume of ejaculate. As an example, when a patient's sperm motility count is below 10 million motile sperm per milliliter of ejaculate and/or at least about 50% of the patient's total sperm count is non-motile and/or, for example, if their ejaculate volume is low (e.g. less than about 1 milliliter), the patient may be considered a candidate for infertility treatment.

Botulinum toxin A is known to block the release of acetylcholine at the nerve ending of striated and smooth muscle causing a temporary, reversible muscle relaxation and further effects. For a detailed discussion of botulinum toxin and its prior uses, reference is made to the background sections of U.S. Pat. Nos. 7,468,189; 7,468,188 and 7,494,654, all hereby incorporated by reference in their entireties.

Administration and techniques for botulinum toxin administration to the prostate are well known in the literature, including the use of botulinum toxin as an intraprosatic injection to treat benign prostate hyperplasia. A non-exhaustive list of examples of such administration can be found, for example, in U.S. Pat. No. 6,365,164, issued Apr. 2, 2002, and in “The use of botulinum toxin in men with benign prostatic hyperplasia”, by Rusnack Susan R and Kaplan Steven A. Rev Urol 2005 Fall;7(4):234-6 and “The application of botulinum toxin in the prostate” by Chuang Yao-Chi and Chancellor Michael B. J Urol 2006; 176(6):2375-82, all herein incorporated by reference in their entireties.

What is needed therefore is a simple method by which ejaculate can be improved in a patient in need thereof, whereby prostatic fluid in the patient's ejaculate is increased.

### SUMMARY

In accordance with one aspect of the invention, a method for increasing ejaculate volume and improvement of the ejaculate of a patient in need thereof is provided, where the method comprises a step of local administration of a therapeutically effective amount of a Clostridial neurotoxin to a prostate of the patient, thereby increasing ejaculate volume of the patient. In particular embodiments, this may include local administration of the therapeutic amount of Clostridial neurotoxin to the prostate via transurethrally or transperineally or transrectally routes and in particular instances, for example when administered via transperineally or transrectally, the method can further comprise the step of utilizing ultrasound guidance during administration to the prostate. A particularly useful Clostridial neurotoxin is a botulinum neurotoxin. Exemplary useful botulinum neurotoxins include botulinum neurotoxin types A , B, C, D, E, F and G.

In some embodiments, the botulinum neurotoxin administered is in an amount of from about 1 unit to 20,000 units, for example. In a specific embodiment, the botulinum neurotoxin is a type A or type B botulinum neurotoxin, and is administered in an amount of from about 1 unit to 3000 units or from about 100 to about 10,000 units, respectively. In a preferred embodiment, botulinum neurotoxin type A is utilized.

Local administration of the therapeutic amount of the Clostridial neurotoxin to the prostate of the patient can be accomplished by, but is not limited to, injection of the Clostridial neurotoxin into the prostate. In particular instances, utilization of ultrasound guidance in conjunction with injection of the Clostridial neurotoxin is helpful in order to visualize the prostate, for example, as well as the administration instruments (e.g. needle) during the administration procedure. In a specific example, local administration of the therapeutic amount of the Clostridial neurotoxin to the prostate of the patient is accomplished via injection into at least one lobe of the prostate and/or can also be accomplished via injection into at least the transition zone of the prostate.

Additionally, a method for increasing ejaculate volume to increase viability of sperm contained therein, in a patient in need thereof, the method comprising the step of local administration of a therapeutically effective amount of a botulinum neurotoxin to a prostate of the patient, thereby increasing ejaculate volume and increasing viability of sperm contained therein, is also presented. The botulinum neurotoxin is preferably selected from the group consisting of botulinum neurotoxin types A, B, C, D, E, F and G. More preferably, the neurotoxin is a botulinum neurotoxin type A and is administered via injection to the prostate of the patient. In particular examples, the amount of botulinum neurotoxin type A administered to the prostate is from about 1 unit to about 2500, more preferably from about 100 units to about 1000 units of neurotoxin type A, the amount to be administered being determined by the attending medical professional, of course. Varying volumes of compositions containing the botulinum neurotoxin can be administered to the prostate of a patient in need thereof. In a particular example, botulinum neurotoxin is administered to the prostate via introduction of about 1 milliliter to about 40 milliliters of a botulinum neurotoxin containing composition to the prostate, preferably a botulinum neurotoxin type A. Preferably, from about 0.5 milliliter to about 10 milliliters of a botulinum neurotoxin containing composition is administered to the prostate in accordance with the present disclosure.

In some instances, multiple sessions of administration of a therapeutically effective amount of a botulinum neurotoxin to the prostate are under taken over a period of time, such as about every two to three months, or about three months after an initial administration to the prostate, or about every three or five or six or nine months, for example. Accordingly and in one example, additional administration of botulinum neurotoxin to the prostate of the patient in need thereof can be performed least about 2 months or about 3 months after an initial administration of botulinum neurotoxin to the prostate.

In a particular example, a method for increasing ejaculate volume to increase viability of sperm contained therein, in a patient in need thereof is provided, the method comprising the step of local administration of a therapeutically effective amount of a botulinum neurotoxin to a prostate of the patient, where the patient's sperm motility count is below 10 million motile sperm per milliliter of ejaculate or at least about 50% of the patient's total sperm count is non-motile, to thereby increase ejaculate volume and increasing viability of sperm contained therein. In a particular embodiment, botulinum neurotoxin is type A or B, is administered via injection into the prostate in an amount of from about 1 to about 1500 units or from about 50 to about 15,000 units, respectively, and in a particular instance, the administration is to at least one lobe of the prostate.

Not wishing to be bound by theory, it can be hypothesized that administration of a botulinum neurotoxin to a prostate of a patient in need thereof can result in relaxation of the periurethral smooth muscles of the prostate and thus could contribute to a more pronounced excretion of prostatic fluid from the glandular positions of the prostate, leading to an increase in the volume of the ejaculate. This ejaculate would then contain more prostatic fluid than an ejaculate from men not having had intraprostatic injections of botulinum neurotoxin, and thus can lead to increased sperm motility and/or quality.

“About” means approximately or nearly and in the context of a numerical value or range set forth herein means ±10% of the numerical value or range recited or claimed.

“Botulinum toxin” means a botulinum neurotoxin as either pure toxin (i.e. about 150 kDa weight molecule) or as a complex (i.e. about 300 to about 900 kDa weight complex comprising a neurotoxin molecule and one or more associated non-toxic molecules), and excludes botulinum toxins which are not neurotoxins such as the cytotoxic botulinum toxins C2 and C3, but can include recombinantly made, hybrid, modified, and chimeric botulinum toxins.

The administration of neurotoxin, such as botulinum neurotoxin, to the prostate is not meant to alter or affect spermatogenesis (i.e. clonal expansion of spermatogonia and subsequent maturation that eventually gives rise to mature sperm), but rather to provide increased prostatic fluid in an ejaculate/increased ejaculate, and thus result in increased/improved characteristics of mature sperm found in the ejaculate. Exemplary improvements can be improved viability and/or motility of sperm contained in the ejaculate as a result of increased volume/increased prostatic fluid in the ejaculate, in accordance with the teachings of the present disclosure.

### DESCRIPTION

The methods disclosed herein are based upon the administration of a neurotoxin, preferably a Clostridial neurotoxin, more preferably a Clostridial botulinum toxin, most preferably a botulinum toxin type A, to the prostate of a patient for which an increase in prostatic fluid and/or ejaculate volume is desired. For example, a patient having a total ejaculate volume of less than about 4 milliliters may be a candidate for the methods herein described for local administration of a botulinum toxin to his prostate in order to increase his ejaculate volume.

In one embodiment, from about 1 to about 2500 units of a botulinum toxin type A may be locally administered to the prostate utilizing known transurethrally or transperineally or transrectally routes. The use of anesthetic during a particular procedure is left to the attending physical/medical professional to determine. Preferably, the lowest therapeutically effective dosage will be administered to the patient. The lowest therapeutic dosage is that dosage which results in the desired effect on the patient's prostate and ejaculate of the patient to which the neurotoxin is administered. Methods for assessing or quantifying the effect of a toxin on a prostate are well known, as is the measurement of ejaculate volume and sperm count and motility, as determined by those skilled in the art.

In patients where their sperm motility count is below 10 million motile sperm per milliliter of ejaculate or at least about 50% of the patient's total sperm count is non-motile, an increase in prostatic fluid can provide for a better environment (more ejaculate/per sperm) and thus the reduced numbers of sperm can have a better chance and be more in a more motility-conducive/friendly environment and thus have a better chance of surviving and eventually finding and penetrating an egg.

The amount of the Clostridial toxin administered according to a method within the scope of the disclosed invention can vary according to the particular characteristics of the patient being treated, including the volume of ejaculate, size of the prostate and other various patient variables including patient size, weight, age, and responsiveness to therapy. To guide the practitioner and as only a general example, typically, no less than about 1 unit and no more than about 100 units of a botulinum toxin type A (such as BOTOX®) is administered per injection site (i.e. to each prostate injection site), per patient treatment session. For a botulinum toxin type A such as DYSPORT® no less than about 2 units and no more about 200 units of the botulinum toxin type A are administered per injection site, per patient treatment session. For a botulinum toxin type B such as MYOBLOC®., no less than about 40 units and no more about 2000 units of the botulinum toxin type B are administered per injection site, per patient treatment session. As one example, administration can include injection of about a total of about 200 units of a botulinum toxin type A, in about a 4 mL volume of liquid (utilizing an appropriate vehicle, such as sterile water or 0.9% non-preserved sterile saline, for example) equally divided into each lateral prostatic lobe (e.g. 2 mL containing about 100 units at two sites per lobe) via a transrectal route of administration.

Although examples of routes of administration (e.g. transurethrally or transperineally or transrectally routes) and the use of ultrasound visualization and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). As an example, the route and dosage for administration of a Clostridial neurotoxin, preferably a botulinum toxin according to the present disclosure, can be selected based upon criteria such as the solubility characteristics of the neurotoxin chosen as well as the lack of (low) ejaculate volume of the patient. Exemplary methods for administration of botulinum toxin to the prostate are known in the art, examples of which can be found in “The potential and promise of using botulinum toxin in the prostate gland” by Chuang Y-C et al. BJU Int 2006;98(1):28-32 and in “The application of botulinum toxin in the prostate” by Chuang Yao-Chi and Chancellor Michael B. J Urol 2006;176(6):2375-82 and references cited therein, all herein incorporated by reference in their entirety.

As stated above, administration of botulinum toxin is thought to result in relaxation of the periurethral smooth muscles. This relaxation could also contribute to a more pronounced excretion of prostatic fluid from the glandular parts of the prostate leading to an increase in the volume of the ejaculate. This ejaculate would then contain more prostatic fluid than an ejaculate from men without intraprostatic injections of botulinum toxin, such as botulinum toxin type A or B, and thus lead to an increased sperm motility and or quality. It is to be understood that typically and as one factor for treatment, having an ejaculate volume of less than 3 milliliters can be indicative of candidacy for intraprostatic administration of a botulinum toxin, as herein disclosed, however the methods and teachings of the present invention are by no means limited to patients having an ejaculate volume of less than 3 milliliters.

Examples

The following non-limiting examples provide those of ordinary skill in the art with exemplary methods to treat conditions within the scope of the present invention and are not intended to limit the scope of the invention.

### EXAMPLE 1

A 42 year-old male presents at his doctor's office and reports that after three years of attempting to impregnate his wife, they remain childless. The doctor conducts a full physical and orders analysis of the patient's ejaculate. Tests show that the patient's volume of ejaculate is only about 1.5 milliliters. The doctor decides that local administration of a botulinum neurotoxin to the patient's prostate may result in an increase in ejaculate volume and thus make it more likely for the patient to impregnate his wife.

The doctor proceeds to administer 200 units of botulinum A toxin (BOTOX®) transrectally into the prostate with the appropriate antibiotic coverage.

One injection into each of the lateral lobes (100 units per injection site, for a total of 200 units) are made. The patient subsequently presents at the doctor's office and is able to produce ejaculate at a volume of about 4 milliliters and proceeds to successfully impregnate his wife.

### EXAMPLE 2

A 26 year-old male presents at his doctor's office, reporting that after five years of attempting to have children, he and his wife remain childless. The doctor accordingly orders up an analysis of the patient's ejaculate and the tests reveal that the patient's sperm motility count is below 10 million motile sperm per milliliter of ejaculate and that his ejaculate is of a small volume, here 1 milliiter. The doctor decides to administer 150 units of a botulinum toxin type A (BOTOX®) transperieneully into each of the lateral lobes (150 units of botulinum toxin type A in 2 milliliters of unpreserved sterile normal saline (0.9% sodium chloride)) into each lobe of the prostate. The patient subsequently reports to the doctor's office about 2 months later and the patient is ordered to provide another sample of ejaculate and additional test show that a greater percentage of his sperm appear more motile and the total volume of ejaculate is now 3 milliliters. The doctor, happy with these results, proceeds to administer a second dose of botulinum toxin to the patient, and the patient can subsequently report 3 months later that his wife is pregnant.

### EXAMPLE 3

A 56 year-old male presents at his doctor's office, and is working on his second marriage. He is reporting that after five years of attempting to have children, he and his younger wife remain childless. The doctor accordingly orders up an analysis of the patient's ejaculate and the tests reveal that the patient's sperm motility count is below 10 million motile sperm per milliliter of ejaculate and that his ejaculate is of a small volume, here 1 milliliter. The doctor decides to administer 150 units of a botulinum toxin type A (BOTOX®) transperienally into each of the lateral lobes (150 units of botulinum toxin type A in 2 milliliters of unpreserved sterile normal saline (0.9% sodium chloride)) into each lobe of the prostate under the appropriate antibiotic coverage. The patient subsequently reports to the doctor's office about 2 months later and the patient is ordered to provide another sample of ejaculate and additional test show that a greater percentage of his sperm appear more motile and the total volume of ejaculate is now 3 milliliters. The doctor, happy with these results, proceeds to administer a second dose of botulinum toxin to the patient, about 6 months later and the patient can subsequently report 3 months later that his wife is pregnant.

As can be seen, patients with a low sperm motility and or decreased sperm quality could potentially benefit from this treatment thus enhancing their ability to actually father a child via intercourse in contrast to having to participate in costly in-vitro fertilization.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variations of exemplary embodiments will become apparent to those of ordinary skill in the art upon reading the foregoing description. The inventor expects skilled artisans to employ such variations as appropriate, and the inventors intend for the invention to be practiced otherwise than specifically described herein. For example, particular ejection volumes and injection particulars such as number of administration sites and locations of administration to the prostate useful in accordance with the teachings of the present disclosure are considered to be within the scope of the present invention. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for increasing ejaculate volume of a patient in need thereof, the method comprising the step of local administration of a therapeutically effective amount of a Clostridial neurotoxin to a prostate of said patient, thereby increasing ejaculate volume of the patient.

2. The method of claim 1, wherein local administration of said therapeutic amount of Clostridial neurotoxin to the prostate is accomplished transurethrally or transperineally or transrectally.

3. The method of claim 2 further comprising the step of utilizing ultrasound guidance.

4. The method of claim 1, wherein the Clostridial neurotoxin is a botulinum neurotoxin.

5. The method of claim 4, wherein the botulinum neurotoxin is selected from the group consisting of botulinum neurotoxin types A , B, C, D, E, F and G.

6. The method of claim 1, wherein the Clostridial neurotoxin is a botulinum neurotoxin and is administered in an amount of from about 1 unit to 20,000 units.

7. The method of claim 1, wherein the Clostridial neurotoxin is botulinum neurotoxin type A or type B, and is administered in an amount of from about 1 unit to 3000 units or from about 100 to about 10,000 units, respectively.

8. The method of claim 1, wherein the Clostridial neurotoxin is botulinum neurotoxin type A.

9. The method of claim 1, wherein local administration of the therapeutic amount of the Clostridial neurotoxin to the prostate of the patient is accomplished via injection of the Clostridial neurotoxin into the prostate.

10. The method of claim 9, further comprising the step of utilizing ultrasound guidance in conjunction with injection of the Clostridial neurotoxin in order to visualize the prostate.

11. The method of claim 1, wherein local administration of the therapeutic amount of the Clostridial neurotoxin to the prostate of the patient is accomplished via injection into at least one lobe of the prostate.

12. The method of claim 1, wherein local administration of the therapeutic amount of the Clostridial neurotoxin to the prostate of the patient is accomplished via injection into at least the transition zone of the prostate.

13. A method for increasing ejaculate volume to increase viability of sperm contained therein, in a patient in need thereof, the method comprising the step of local administration of a therapeutically effective amount of a botulinum neurotoxin to a prostate of the patient, thereby increasing ejaculate volume and increasing viability of sperm contained therein.

14. The method of claim 13, wherein the botulinum neurotoxin is selected from the group consisting of botulinum neurotoxin types A, B, C, D, E, F and G.

15. The method of claim 13, wherein the botulinum neurotoxin is botulinum neurotoxin type A and is administered via injection to the prostate of the patient.

16. The method of claim 15, wherein an amount of botulinum neurotoxin type A administered to the prostate is from about 1 unit to about 2500 units.

17. The method of claim 13, wherein the botulinum neurotoxin type A is administered to the prostate via introduction of about 1 milliliter to about 40 milliliters of a botulinum neurotoxin containing composition to the prostate.

18. The method of claim 13, further comprising additional administration of botulinum neurotoxin to the prostate of the patient least 2 months after an initial administration of botulinum neurotoxin to the prostate.

19. A method for increasing ejaculate volume to increase viability of sperm contained therein, in a patient in need thereof, the method comprising the step of local administration of a therapeutically effective amount of a botulinum neurotoxin to a prostate of the patient, wherein the patient's sperm motility count is below 10 million motile sperm per milliliter of ejaculate or at least about 50% of the patient's total sperm count is non-motile, thereby increasing ejaculate volume and increasing viability of sperm contained therein.

20. The method of claim 19, wherein the botulinum neurotoxin is type A or B, and is administered via injection into the prostate in an amount of from about 1 to about 1500 units or from about 50 to about 15,000 units, respectively.

21. The method of claim 20, wherein the administration is to at least one lobe of the prostate.
